(19) United States
(12) Patent Application Publication
(10) Pub. No.: US 2021/0106384 A1
(43) Pub. Date: Apr. 15, 2021

(54) WHITENING AND REJUVENATING LASER HAIR REMOVER

(71) Applicant: Shenzhen Fangyuan Tongchuang Technology Co., Ltd., Shenzhen (CN)

(72) Inventor: Shuai Wang, Fenyang (CN)

(21) Appl. No.: 16/599,136

(22) Filed: Oct. 11, 2019

(51) Int. Cl. A61B 18/20; A61N 5/06

(52) U.S. Cl. CPC A61B 18/203; A61B 2018/00023; A61N 5/0616

(57) ABSTRACT

The invention discloses a whitening and rejuvenating laser hair remover in technical field of hair remover, including main device and flash head integrated. The flash head, with laser emitter port is set at the front end of the body. The main device is composed of body and handle. There are LED display screen on the side of the body, power button on one side of the LED display screen, cooling fan at the back end of the body, photometric gear button on the handle, quartz lamp tube and printed circuit board installed inside the body. The quartz lamp tube is corresponding to the laser emitter port, and the printed circuit board is composed of the power supply module, laser emission module, laser ranging module, photometric adjustment module and laser counting module. The invention of whitening and rejuvenating laser hair remover is portable, with a long service life, stable performance, less stimulation to the skin and better hair removal effect. New collagen fiber and elastic fiber are produced by acting on skin, thereby enhancing skin elasticity, shrinking pore, with effect of whitening and rejuvenating.

### TECHNICAL FIELD

The invention relates to the technical field of hair remover, especially a whitening and rejuvenating laser hair remover.

### BACKGROUND TECHNOLOGY

Traditional hair removal methods generally include dense wax, razor, cream, etc., which is time-consuming and laborious. At present, there are various laser hair removers on the market. By close irradiation of hair follicle melanin with pulsed light, the hair follicle can be heated and inactivated, to delay hair growth. Traditional laser hair removal needs instantaneous high-energy burning of hair follicles. When the laser hair remover is too close to the skin, it will over-stimulate the skin, burn the hair and caused pain risks, and infection to the hair removal site, which brings bad experience to users. When the laser hair remover is relatively distant from the skin, the effect of hair removal is not ideal.

### SUMMARY OF THE INVENTION

To solve problems of excessive skin irritation, pain or risk, and infection of hair removal site caused by the laser hair remover in the available technology, a whitening and rejuvenating laser hair remover is invented.

For the said purpose, the following technical scheme is applied in the invention:

A whitening and rejuvenating laser hair remover, including main device and flash head, is characterized in that the flash head, with laser emitter port is set at the front end of the body. The main device is composed of body and handle. There are LED display screen on the side of the body, power button on one side of the LED display screen, cooling fan at the back end of the body, photometric gear button on the handle, quartz lamp tube and printed circuit board installed inside the body. The quartz lamp tube is corresponding to the laser emitter port, and the printed circuit board is composed of the power supply module, laser emission module, laser ranging module, photometric adjustment module and laser counting module. The invention of whitening and rejuvenating laser hair remover is portable, with a long service life, stable performance, less stimulation to the skin and better hair removal effect. It acts on the skin to produce new collagenous fiber and elastic fiber, thus enhancing the skin elasticity, shrinking pores, and presenting the effect of whitening and rejuvenating.

Preferably, the printed circuit board is electrically connected with the power button, LED display screen, quartz tube and photometric gear button, to control the function of the hair remover.

Preferably, the cooling vent corresponding to cooling fan is arranged on the rear shell of the body, to discharge the heat produced by the hair remover.

Preferably, the photometric gear button is provided with 5 gears, to adjust the appropriate luminosity according to the skin condition, thus avoiding damage caused by excessive irradiation on the skin.

Preferably, with the emitting area, flash times and wavelength of the laser emission module of 3.2 cm², 500,000, and 470-1,200 nm, the flash head can adapt to multiple body parts, with long service period, and good hair removal effect.

Preferably, the socket is set at the bottom of the handle.

Preferably, the shell of the main device is made from ABS materials, with excellent impact resistance, heat resistance, low-temperature resistance, chemical resistance and electrical performance.

Compared with available technology, the invention is a whitening and rejuvenating laser hair remover, with the following beneficial effects:

1 The printed circuit board is electrically connected with the power button, LED display screen, quartz tube and photometric gear button, to control the function of the hair remover.
2 The cooling vent corresponding to cooling fan is arranged on the rear shell of the body, to discharge the heat produced by the hair remover.
3 The photometric gear button is provided with 5 gears, to adjust the appropriate luminosity according to the skin condition, thus avoiding damage caused by excessive irradiation on the skin.
4 With the emitting area, flash times and wavelength of the laser emission module of 3.2 cm², 500,000, and 470-1,200 nm, the flash head can adapt to multiple body parts, with long service period, and good hair removal effect.
5 The shell of the main device is made from ABS materials, with excellent impact resistance, heat resistance, low-temperature resistance, chemical resistance and electrical performance.

The invention, a whitening and rejuvenating laser hair remover, is portable and convenient for use, with long service time, stable performance, high safety factor, less stimulation to skin during hair removal, and better hair removal effect. New collagen fiber and elastic fiber are produced by acting on skin, thereby enhancing skin elasticity, shrinking pore, with effect of whitening and rejuvenating.

### DESCRIPTION OF THE DRAWINGS

FIG. 1 shows a schematic diagram of the three-dimensional structure of the invention.

FIG. 2 shows a schematic diagram of the connection structure between the printed circuit board module and various modules of the hair remover.

Figure: 1 main device, 2 body, 3 handle, 4 LED display screen, 5 power button, 6 cooling vent, 7 photometric gear button, 8 power socket, 9 flash head, 10 laser emitter port, 11 irradiation key and 12 flash key.

### Specific Implementation Mode

Combined with the drawings in the embodiment of the invention, the following text will clearly and completely describe the technical solution in the embodiment of the invention. Obviously, the embodiment described is merely partial embodiment in the invention.

In the description of the invention, it is to be understood that the orientation or position relation indicated by terms “up”, “down”, “front”, “back”, “left”, “right”, “top”, “bottom”, “inside” and “outside” is based on that shown in the drawings, which is intended to facilitate the description of the invention and simplify the description, instead of indicating or implying that devices or elements referred to must have a specific orientation, be constructed and operated in a specific orientation, and therefore cannot be understood as a limitation of the invention.

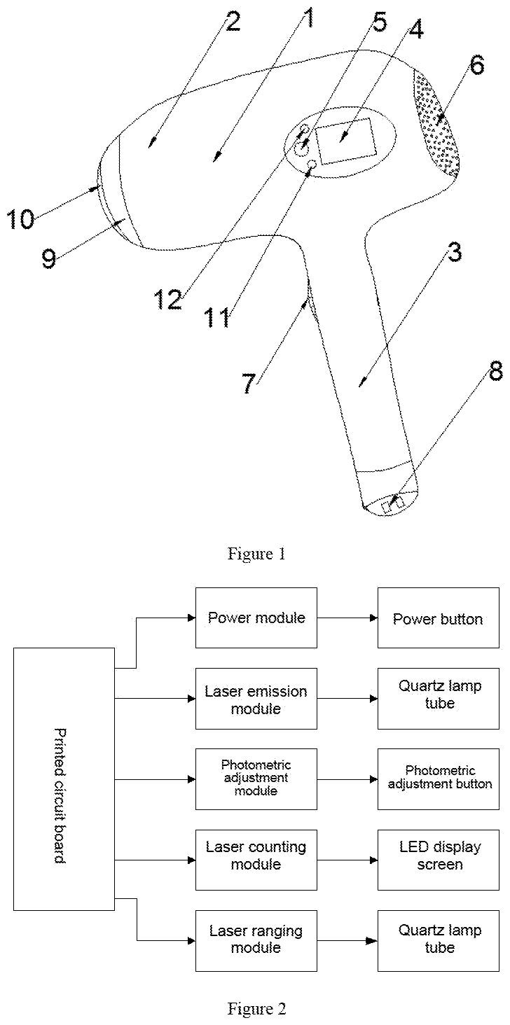

With reference to FIG. 1-2, a whitening and rejuvenating laser hair remover, including main device 1 and flash head 9 integrated. The flash head 9, with laser emitter port 10 is set at the front end of the body 2. The main device 1 is composed of body 2 and handle 3. There are LED display screen 4 on the side of the body 2, power button 5, flash key 12 and irradiation key 11 on one side of the LED display screen, cooling fan at the back end of the body 2, photometric gear button 7 on the handle 3, quartz lamp tube and printed circuit board installed inside the body. The quartz lamp tube is corresponding to the laser emitter port 10. The ultraviolet rays are effectively filtered through the quartz lamp tube, without any illumination side effects. The printed circuit board is composed of the power supply module, laser emission module, laser ranging module, photometric adjustment module and laser counting module. The printed circuit board is electrically connected with the power button 5, LED display screen 4, quartz tube and photometric gear button, to control the finction of the hair remover. The cooling vent corresponding to cooling fan is arranged on the rear shell of the body, to discharge the heat produced by the hair remover. The photometric gear button is provided with 5 gears, to adjust the appropriate luminosity according to the skin condition, thus avoiding damage caused by excessive irradiation on the skin. With the emitting area, flash times and wavelength of the laser emission module of 3.2 cm², 500,000, and 470-1,200 nm, the flash head 9 can adapt to multiple body parts, with long service period, and good hair removal effect. The socket 8 is set at the bottom of handle 2. The shell of main device 1 is made from ABS materials, with excellent impact resistance, heat resistance, low-temperature resistance, chemical resistance and electrical performance.

The working principle of the invention is that the quartz lamp tube is powered by alternating current. By irradiation of hair follicle melanin with intense pulsed light generated by the laser emission module, the hair follicle can be heated and inactivated, to delay hair growth. New collagen fiber and elastic fiber are produced by acting on skin, thereby enhancing skin elasticity, shrinking pore, with effect of whitening and rejuvenating.

Before use, first, shave the skin surface hair, wear safety goggles, avoid strong light flashing into their eyes, hold the handle at position 3, and press power button 5. In case of distant irradiation, the hair remover detects that the distance is too far, the quartz lamp tube will not be powered on. In case of close irradiation, the quartz lamp tube is powered on twice in 1.5 s, press flash button 12 for a long time, so that the hair remover is in continuous flash mode, that is, the hair remover can be used normally. Press irradiation key 11 to fully fit the laser emitter port 10 on the flash head 9 with the skin. Press the photometric gear button 7 to set the light intensity. The hair remover is provided with 5 gears of illumination intensity. It is recommended that the hair remover should be used from gear 1 for the first time. According to the specific sensation of the skin, the gear can be increased gradually. The higher the gear is, the better the effect of hair removal treatment is. The laser counting module is used to record the number of photons remaining in use and display on the LED display screen 4. The hand-held laser hair remover of the invention has a total of 500,000 flash times. The whole body hair removal only needs about 2,000 flash times, and it can restrain the hair growth for 8 times, namely, about 16,000 flashes in the whole period. 500,000 flash times can be used for 10-15 people to remove the hair all over the body, which is durable, with a long service period. Note: In case of scars on the body surface or skin with light sensitization, the irradiated skin that is slightly reddish can be subject to icing.

What is said above is only a better implementation mode of the invention, but the protection scope of the invention is not limited to this. The equivalent replacement or change made by any technician who is familiar with this technical field within the technical scope disclosed by the invention according to technical solution and its invention concept of the invention falls in the protection scope of the invention.

1. A whitening and rejuvenating laser hair remover, including main device and flash head, is characterized in that the flash head, with laser emitter port is set at the front end of the body. The main device is composed of body and handle. There are LED display screen on the side of the body, power button, flash key, and irradiation key on one side of the LED display screen, cooling fan at the back end of the body, photometric gear button on the handle, quartz lamp tube and printed circuit board installed inside the body. The quartz lamp tube is corresponding to the laser emitter port, and the printed circuit board is composed of the power supply module, laser emission module, laser ranging module, photometric adjustment module and laser counting module. The invention of whitening and rejuvenating laser hair remover is portable, with a long service life, stable performance, less stimulation to the skin and better hair removal effect. It acts on the skin to produce new collagenous fiber and elastic fiber, thus enhancing the skin elasticity, shrinking pores, and presenting the effect of whitening and rejuvenating.

2. A whitening and rejuvenating laser hair remover as set forth in claim 1 is characterized in that the printed circuit board is electrically connected with the power button, LED display screen, quartz tube and photometric gear button.

3. A whitening and rejuvenating laser hair remover as set forth in claim 1 is characterized in that the cooling vent corresponding to cooling fan is arranged on the rear shell of the body.

4. A whitening and rejuvenating laser hair remover as set forth in claim 1 is characterized in that the photometric gear button has 5 gears.

5. A whitening and rejuvenating laser hair remover as set forth in claim 1 is characterized in that the emitting area, flash times and wavelength of the laser emission module of the flash head are 3.2 cm², 500,000, and 470-1,200 nm.

6. A whitening and rejuvenating laser hair remover as set forth in claim 1 is characterized in that the socket is set at the bottom of the handle.

7. A whitening and rejuvenating laser hair remover as set forth in claim 1 is characterized in that the shell of the main device is made from ABS materials.
